(19) United States
(12) Patent Application Publication
(10) Pub. No.: US 2019/0380965 A1
(43) Pub. Date: Dec. 19, 2019

(54) Quick Water-Dispersible Pharmaceutical Compositions of Flucytosine

(71) Applicants: Hemant N. Joshi, Parsippany, NJ (US); Amitkumar Lad, Dumont, NJ (US)

(72) Inventors: Hemant N. Joshi, Parsippany, NJ (US); Amitkumar Lad, Dumont, NJ (US)

(21) Appl. No.: 16/009,909

(22) Filed: Jun. 15, 2018

(51) Int. Cl. A61K 9/16; A61K 31/513; A61K 31/7048

(52) U.S. Cl. CPC A61K 9/1652; A61K 31/7048; A61K 31/513

(57) ABSTRACT

Quick water-dispersible pharmaceutical compositions of Flucytosine in powder or granule form are provided that allow for ease of preparation of measured-dose, liquid suspensions that are palatable and suitable for pediatric administration. The compositions can include Flucytosine as an active or combination with other anti-fungal agents, suspending agent, sweetener, flavoring agent and other additional excipients. The compositions in granular or powder form are efficiently dissolvable/dispersible in water and can be provided encapsulated within a two-piece dispensing hard capsule or a packet or pouch for easy release.

### TECHNICAL FIELD

The present disclosure relates to quick water dispersing pharmaceutical composition of flucytosine, which features the active ingredient flucytosine with optionally other antifungal agents in granular or powder form that allows an efficient preparation of measured-dose, palatable liquid suspensions suitable for pediatric and/or geriatric dosing and for persons having swallowing difficulties. The powder composition contains a suspending agent having particle size in the range of 177 to 420 microns to obtain homogeneous and stable suspension in a short time. The composition, with a desired particle size of the suspending agent, provides suitable flow properties of the powder and solves the formation of lump while making the suspension.

### BACKGROUND

Flucytosine is 5-fluorocytosine, a fluorinated, pyrimidine which is related to fluorouracil and floxuridine. Flucytosine is an anti-fungal agent and used for serious infections caused by susceptible strains of Candida and/or cryptococcus.

Flucytosine is taken up by fungal organisms via the enzyme cytosine permease. Inside the fungal cell, flucytosine is rapidly converted to 5-fluorouracil (5-FU) by the enzyme cytosine deaminase. Fluorouracil exerts its antifungal activity through the subsequent conversion into several active metabolites, which inhibit protein synthesis by being falsely incorporated into fungal RNA or interfere with the biosynthesis of fungal DNA through the inhibition of the enzyme thymidylate synthetase.

Candida is a genus of yeasts and is the most common cause of fungal infections worldwide. Candida albicans is the most commonly isolated species, and can cause infections (candidiasis or thrush) in humans and other animals [1]. Among Candida species, C. albicans, which is a normal constituent of the human flora, a commensal of the skin and the gastrointestinal and genitourinary tracts, is responsible for most of Candida bloodstream infections (candidemia). Yet, there is an increasing incidence of infections caused by C. glabrata and C. rugosa, which could be because they are frequently less susceptible to the currently used azole antifungals [2]. Other medically important species include C. parapsilosis, C. tropicalis and C. dubliniensis [3].

Cryptococcus are the species that can cause cryptococcal disease, a potentially fatal fungal disease. It is caused by one of two species; Cryptococcus neoformans and Cryptococcus gattii. Cryptococcosis is believed to be acquired by inhalation of the infectious propagule from the environment. Cryptococcosis is a defining opportunistic infection for AIDS, and is the second-most-common AIDS-defining illness in Africa. Other conditions that pose an increased risk include certain lymphomas (e.g., Hodgkin's lymphoma), sarcoidosis, liver cirrhosis, and patients on long-term corticosteroid therapy. In humans, C. neoformans causes three types of infections: Wound or cutaneous cryptococcosis, Pulmonary cryptococcosis and Cryptococcal meningitis.

U.S. Pat. No. 9,314,524 claims topical formulations of flucytosine for treatment and maintenance of fungal infections, particularly vulvovaginal candidiasis [4]. US patent 2009/0068287A1 describes preparation and use of topical flucytosine formulation. The invention provides novel topical formulations of flucytosine designed to allow the active drug to act at the local application area, but which inhibit or moderate transdermal or transmucosal absorption of the drug, thus limiting systemic exposure [5].

The usual dosage of flucytosine is 50 to 150 mg/kg/day administered in divided doses at 6-hour intervals. Flucytosine capsules have been used to treat candida infections such as septicemia, endocarditis and urinary system infections effectively and Cryptococcus infections such as meningitis and pulmonary infections effectively.

Currently, in the United States Flucytosine is sold as the brand name ANCOBAN® or as a generic medicine that is only available in Capsules of 250 mg base or 500 mg base. Table 1 lists the effect of density of material on the fill weight in mg in different size capsules. We observed the density of flucytosine blend to be about 0.7 g/mL. It means, 500 mg dose will fit in size 00 capsule, which is big even for adults. We are also proposing formulation of flucytosine with other antifungal agents. It means, the final fill weight of granules to be filled in the capsules will be higher. If for a formulation, we must use size 000 capsule, even adults won't be able to swallow it. For pediatric dosing, a pharmacist empties the contents of the capsules to a mortar, adds vehicle such as ORA-PLUS®—a commercial product and mixes well. The preparation of suspension is a tedious procedure and may involve loss of a portion of drug during preparation, Suspension media such as ORA-PLUS® is costly. At home or sometimes in the compounding pharmacies, ORA-PLUS® may not be available. It is better to have a ready-to-use flucytosine powder formulation for suspension, already containing suspending agent and other excipients, which is likable by children,

TABLE I
Effect of density of the material on the fill weight in mg in different
size capsules.

| Density, g/mL | Size 1 | Size 0 | Size 00 | Size 000 |
|---|---|---|---|---|
| 0.3 | 150 | 204 | 285 | 411 |
| 0.4 | 200 | 272 | 380 | 548 |
| 0.5 | 250 | 340 | 475 | 685 |
| 0.6 | 300 | 408 | 570 | 822 |
| 0.7 | 350 | 476 | 665 | 959 |
| 0.8 | 400 | 544 | 760 | 1096 |
| 0.9 | 450 | 612 | 855 | 1233 |
| 1.0 | 500 | 680 | 950 | 1370 |
| 1.1 | 550 | 748 | 1045 | 1507 |
| 1.2 | 600 | 816 | 1140 | 1644 |

Thus, there is an unmet need for improved flucytosine formulations that are amenable for dosing to children and persons with swallowing difficulties. One should be able to make the liquid formulation in a home-setting with just water or can be sprinkled on. The present invention provides such improved compositions, methods of preparation and use thereof.

### SUMMARY OF INVENTION

In one embodiment, a water-dispersible pharmaceutical composition of flucytosine is provided comprising:
(i) about 50 weight % to about 95 weight % flucytosine;
(ii) about 0.1 weight. % to about 20 weight % of one or more suspending agents with particle size 177 to 420 microns;
(iii) about 0.1 weight % to about 30 weight % of a diluent(s);
(iv) about 0.01 weight % to about 5 weight % of one or more of natural or artificial or combination of sweetening agents;
(v) optionally about 0.01 weight % to about 5 weight % of a flavoring agent
(vi) optionally up to about 20 weight % of one or more of an additional excipients and adjuvants,
   wherein the sum of (i)-(vi) is 100%; wherein the pharmaceutical composition is in the form of granules or a powder and the pharmaceutical composition is prepared by using the wet granulation method.

In another embodiment, a water-dispersible pharmaceutical composition is provided comprising:
(i) about 10 weight % to about 95 weight % flucytosine;
(ii) about 10 to 95 weight % of another anti-fungal agent or a mixture of anti-fungal agents.
(iii) about 0.1 weight % to about 20 weight % of one or more suspending agents with particle size 177 to 420 microns;
(iv) about 0.1 weight % to about 30 weight % of a diluent(s);
(v) about 0.01 weight % to about 5 weight % of one or more of natural or artificial or combination of sweetening agents;
(vi) optionally about 0.01 weight ° A) to about 5 weight % of a flavoring agent
(vii) optionally up to about 20 weight % of one or more of an additional excipients and adjuvants,
   wherein the sum of (i)-(vii) is 100%; wherein the pharmaceutical composition is in the form of a granule or a powder and the pharmaceutical composition is prepared by using the wet granulation method.

In another embodiment, a water-dispersible pharmaceutical composition is provided comprising:
(viii) about 10 weight % to about 95 weight % flucytosine;
(ix) optionally about 10 to 95 weight % of another anti-fungal agent or a mixture of anti-fungal agents.
(x) about 0.1 weight. % to about 20 weight % of one or more suspending agents with particle size 177 to 420 microns;
(xi) about 0.1 weight % to about 30 weight % of a diluent(s);
(xii) about 0.01 weight % to about 5 weight % of one or more of natural or artificial or combination of sweetening agents;
(xiii) optionally about 0.01 weight % to about 5 weight % of a flavoring agent
(xiv) optionally up to about 20 weight % of one or more of an additional excipients and adjuvants,
   wherein the sum of (i)-(vii) is 100%; wherein the pharmaceutical composition is in the form of a granule or a powder and the pharmaceutical composition is prepared by dry granulation method.

The compositions mask the bitter taste of the Flucytosine and other antifungal agents and allow for efficient dissolution/suspension of the granules or powder in water. In one embodiment, the water-dispersible pharmaceutical compositions comprising Flucytosine are encapsulated within a two-piece releasable hard capsule or a packet or a pouch. The encapsulated forms of the pharmaceutical composition allow for ease of preparation of pediatric or geriatric weight-based doses.

In one embodiment, a process is provided for preparing the water-dispersible pharmaceutical compositions comprising Flucytosine: combining a Flucytosine, a suspending agent(s), and optionally one or more excipients and adjuvants; granulating the combination with water or with a sweetening agent(s) in water or with an adjuvant(s) in water; drying the granules; and optionally, combining the dried granules with flavoring agents, excipients, and adjuvants. In this case, water or water containing a sweetener or an adjuvant is termed as an aqueous phase used for granulation.

In one embodiment, a method is provided for preparing measured-dose liquid suspension of a Flucytosine pharmaceutical composition, the method comprising: releasing a water-dispersible pharmaceutical composition comprising Flucytosine in granules or powder form that is encapsulated within a two-piece releasable hard capsule or a packet or a pouch; and dissolving or suspending a measured-dose of the released granules or powder in water. In another embodiment, due to good flow property of the flucytosine granules or powder, the granules can be sprinkled on food items such as Jell-O, yogurt, apple sauce etc. and administered, to, the patient. When the granules are in a capsule, the capsule can also be administered to a patient as is. Thus, adult patient may take the intact capsule with water, and to pediatric or geriatric patients, powder the capsules can be easily suspended in water before administration or sprinkled on food items.

### DETAILED DESCRIPTION

The present invention features water-dispersible pharmaceutical compositions comprising the active ingredient Flucytosine in granules or powder form that allows an efficient preparation of a measured-dose, palatable liquid suspensions suitable for pediatric or geriatric dosing and for persons having swallowing difficulties. Granules filled in a capsule can also taken with some water as is (without suspending granules/powder in water).

Throughout this specification and the claims, the terms “comprise,” “comprises,” and “comprising” are used in a non-exclusive sense, except where the context requires otherwise. Likewise, the terms “having” and “including” and their grammatical variants are intended to be non-limiting, such that recitation of items in a list is not to the exclusion of other like items that can be substituted or added to the listed items.

The term “swallow” means to cause or allow to pass down the throat.

The term “suspending agent” means a substance that is added to fluids to promote particle suspension or dispersion and reduce sedimentation. A suspending agent increasing the viscosity of the liquid system and slows down the sedimentation of undissolved or suspended particles.

The term “flavoring agent” is defined as the substance that added to give a taste to a formulation by providing a nice flavor.

A “sweetener” is a substance especially one other than sugar that sweetens the formulation.

“Other antifungal agent(s)” is termed as antifungal agent other than flucytosine and has anti-fungal activity.

Throughout this specification and the claims, the terms “dissolution”, “dispersion”, and “suspension” and their grammatical variants are used interchangeably and are intended to mean that a composition is capable of being suspended or dispersed in an aqueous solution and may also be capable of being partially or completely solubilized or dissolved in the aqueous solution.

The term “diluent” is refer as substance which acts as a filler in a formulation to increase weight and improve content uniformity.

The term “granules” is refers to the substance in which primary powder particles are made to adhere to form larger, multiparticle entities. The “Aqueous Phase for granulation” can be plain water, water with a sweetener or water with an adjuvant such as color etc. Granules are prepared using the aqueous phase of granulation followed by drying. This is termed as a “wet granulation” method. One can also make the drug composition by a dry blending method. In this case, the drug and excipients are simply mixed in a blender. The process is termed as “dry blending” or “dry granulation” method.

A suspension is a heterogeneous mixture in which the solute particles do not dissolve, but get suspended throughout the bulk of the solvent, left floating around freely in the medium. The “Aqueous Phase for Formulation” can be plain water, or a juice or a drink.

The term “sprinkle on food” means pouring of small particles/granules from the capsule content on food. The food can be an easy to eat preparations such as Jell-O, yogurt, apple sauce etc.

The term “pouch” refers as small bag or flexible packet filled with granules or powder in it and then sealed.

Flucytosine is available in the United States market in a 250 mg and 500 mg strength formulations (ANCOBON® from Valeant Pharmaceuticals International). ANCOBON® and generic capsules are all immediate-release capsule formulations. Currently, pediatric formulations of Flucytosine are not available. Thus, for dosing by weight for children, prior art Flucytosine contents are taken out from the capsules and added to mortar. A vehicle ORA-PLUS® is added to the mortar and mixed. The preparation of suspension is a tedious procedure. Suspension media such as ORA-PLUS® is costly. The process results in a loss of active pharmaceutical ingredient. In addition, the shelf-life of such a suspension tends to be only 30 days at 2-8° C. protected from light. In many instances, the suspension media ORA-PLUS® may not be easily available in the home setting.

The present invention solves these problems with granular and powder formulations of Flucytosine that can be provided by filling in capsules, packets or pouches. The powder formulation can be added to an aqueous phase for formulation in a measured dose in a measuring cup in order to easily prepare a palatable liquid suspension. The suspended formulations can be administered to children or elderly patients or people with difficulty in swallowing solid dosage forms. The unaltered original capsules (intact capsule) filled with the drug composition are also useful for administration to adults. Additionally, the suspended formulation masks the bitter taste of the Flucytosine optionally with other antifungal agents and allows for efficient dispersion of the granules or powder in water.

Another advantage of the present invention is that the pharmaceutical compositions in the granular and powder form are stable at room temperature for at least 2 years.

The powder and granule pharmaceutical compositions of the present invention dissolve or suspend when added to water quickly. This pharmaceutical composition is therefore termed as “quick water-dispersing”. The term “quick” herein is defined as less than 2 to 3 minutes when shaken gently. In other words, when the granules are added to water, and gently mixed, a suspension is formed in less than 2 to 3 minutes. The “quick water-dispersing” property was achieved by selecting a correct particle size of suspending agent(s) which is the novelty of this invention. The suspension is achieved by simple mixing of powder in water—without use of complex instruments which are available in the home-environment to patients. Flucytosine solubility in water is reported to be 15 g/L or 15 mg/mL. When granules containing 500 mg flucytosine are added to 10 mL water, about 150 mg drug dissolves in water and about 350 mg drug stays in a suspended form. After reaching the gastro-intestinal tract, the remaining drug dissolves. The pharmaceutical compositions according to the present invention provide for an immediate release of the Flucytosine from the dosage form.

A typical example herein describes preparation of a water-dispersible Flucytosine granule pharmaceutical composition. In one aspect of the present invention, quick water-dispersible pharmaceutical compositions are provided comprising:
(i) about 50 wt. % to about 95 weight % Flucytosine;
(ii) about 0.1 wt. % to about 20 weight % of one or more of a suspending agent(s) with particle size of 177 to 420 microns for the production of a homogenous and stable suspension;
(iii) about 0.1 wt % to about 30 weight % of a diluent(s);
(iv) about 0.01 wt. % to about 5 weight % of one or more of natural, artificial or combination of sweetening agents;
(v) optionally about 0.01 wt. % to about 5 weight % of a flavoring agent
(vi) optionally up to about 20 weight % of one or more of an additional excipients and adjuvants,
   wherein the sum of (i)-(vi) is 100%; wherein the pharmaceutical composition is in the form of granules or a powder and the pharmaceutical composition is prepared by using the wet granulation method.

In another example water-dispersible pharmaceutical compositions are provided comprising:
(i) about 10 wt. % to about 95 weight % combination of Flucytosine
(ii) about 10 wt. % to about 95 weight % other antifungal agent or a combination of other antifungal agents.
(iii) about 0.1 wt. % to about 20 weight ° A of one or more of a suspending agent(s) with particle size of 177 to 420 micron;
(iv) about 0.1 wt. % to about 10 weight % of a diluent(s);
(v) about 0.01 wt. % to about 5 weight % of one or more of natural, artificial or combination of sweetening agents;
(vi) optionally about 0.01 weight % to about 5 wt. % of a flavoring agent
(vii) optionally up to about 20 weight % of one or more of an additional excipients and adjuvants,
   wherein the sum of (i)-(vii) is 100%; wherein the pharmaceutical composition is in the form of granules or a powder and the pharmaceutical composition is prepared by using the wet granulation method.

In another example water-dispersible pharmaceutical compositions are provided comprising:
(i) about 10 wt. % to about 95 weight % combination of Flucytosine
(viii) Optionally about 10 wt. % to about 95 weight % other antifungal agent or a combination of other antifungal agents.
(ix) about 0.1 wt. % to about 20 weight % of one or more of a suspending agent(s) with particle size of 177 to 420 microns;
(x) about. 0.1 wt. % to about 10 weight % of a diluent(s);
(xi) about 0.01 wt. % to about 5 weight % of one or more of natural, artificial or combination of sweetening agents;
(xii) optionally about 0.01 weight ° A to about 5 wt. % of a flavoring agent
(xiii) optionally up to about 20 weight % of one or more of an additional excipients and adjuvants,
   wherein the sum of (i)-(vii) is 100%; wherein the pharmaceutical composition is in the form of granules or a powder and the pharmaceutical composition is prepared by using the dry granulation method.

Suitable suspending agents include, but are not limited to, one or a combination of microcrystalline cellulose, colloidal silicon dioxide, sodium carboxymethyl cellulose (NaCMC), hydroxypropyl methylcellulose, hydroxylpropyl cellulose, methyl cellulose, alginates, acacia, xantham gum, carbomer, bentonite acrylic acid polymer, methacrylate polymers Eudragits). The average molecular weight of the NaCMC is about 10,000 to 200,000.

The pharmaceutical compositions can be in the form of a hard-shell capsule. The pharmaceutical compositions can be provided in a dosage unit of 50 mg to 500 mg of Flucytosine. The dosage unit can be 250 mg and 500 mg of Flucytosine as currently sold in the United States. Using this technique, one can make capsules or pouches with more than 500 mg Flucytosine per unit dosage form.

The pharmaceutical compositions in the powder or granular form can be stable at room temperature for at least 2 years.

It is one objective of the present invention to provide pharmaceutical compositions comprising Flucytosine that can be prepared efficiently in measured-dose liquid suspensions that are palatable for oral administration, Thus, in one feature of the present invention, the water-dispersible pharmaceutical compositions comprising Flucytosine are provided encapsulated within a two-piece releasable hard capsule. Suitable capsules are known in the art and including capsules that consist essentially of hydroxypropyl methylcellulose and capsules that consist essentially of gelatin.

In one feature of the present invention, a method is provided that includes releasing the water-dispersible pharmaceutical composition encapsulated within a two-piece releasable hard capsule and dissolving or suspending a measured dose of the released granules or powder in an aqueous phase or medium such as water.

In one feature of the present invention, a method is provided that includes releasing the water-dispersible pharmaceutical composition encapsulated within a packet or a pouch and dissolving or suspending a measured dose of the released granules or powder in an aqueous phase or medium such as water.

Other anti-fungal agents which can be coadministered with flucytosine include, but, are not limited to, one or a combination of amphotericin B, ketoconazole, itraconazole, fluconazole, voriconazole, posaconazole, isavuconazole, clotrimazole, micafungin, anidulafungin, griseofulvin, terbinafine, efinaconazole, econazole, miconazole, terconazole, butoconazole, ciclopirox olamine, tolnaftate, nystatin and many more.

A sweetening agent and/or a flavoring agent can be included in the pharmaceutical compositions of the present invention to further increase the palatability of the liquid suspensions. The sweetening agents are preferably non-acidic sweetening agents. Suitable sweetening agents include, but are not limited to, one or a combination of fructose, glucose, sucrose, erythritol, sorbitol, mannitol, xylitol, honey, maple syrup, sucralose, saccharin, aspartame, neotame, sucralose, and advantame.

Natural or artificial flavoring agent can be included in the pharmaceutical composition of the present invention. The flavors include raspberry, strawberry, mint, banana, grape, cherry, orange, and fruit punch. Other commonly used flavors in the pharmaceutical industry can also be used in this formulation.

Regarding the diluents that can be included in the water-dispersible pharmaceutical compositions of the present invention, suitable diluents include, but are not limited to, one or a combination of corn starch, hydrolyzed corn starches, partially pregelatinized starches, anhydrous lactose, lactose monohydrate, sugar alcohols, sorbitol, xylitol, and mannitol.

Additional excipients and adjuvants can be included in the pharmaceutical compositions of the present invention, and are selected from binders, carriers, lubricants, flow control agents such as glidant, and crystallization retarders.

### Effect of Particle Size on Dissolution of Sodium CMC

Different grades of sodium CMC were used for dissolution purpose. All grades were passed through different number of sieves, which correspond to different particle sizes. Table 2 lists the opening in inches and corresponding particle size in microns for representative sieves. Sieves were placed on top of each other and the drug substances was allowed to pass through. Fractions were collected on different sieves. Particle fractions were collected above 420-microns, between 177-420 microns and less than 177 microns. Same amounts of different fractions of sodium CMC were added to 10 mL of water and stirred for 1 minute to check dissolution of sodium. CMC. It was found that particles collected between 177-420 microns dissolved fastest and easily compared to all other particle sizes. Sodium CMC tends to form lumps when exposed to water and then it is hard to disperse. Thus, it is very crucial in the formulation to have a suspending agent with a desire particle size, observed to be 177 to 420 microns, to obtain a stable suspension in which the drug particles remain suspended for a certain period.

TABLE 2
Sieve size and particle size chart

| Sieve size | Opening in inches | Particle size in Microns |
|---|---|---|
| 40 | 0.0167 | 420 |
| 80 | 0.0071 | 177 |
| 200 | 0.0030 | 74 |
| 400 | 0.0015 | 38 |

The following examples have been included to provide guidance to one of ordinary skill in the art for practicing representative embodiments of the presently disclosed subject matter. Considering the present disclosure and the general level of skill in the art, those of skill can, appreciate that the following examples are intended to be exemplary only and that numerous changes, modifications, and alterations can be employed without departing from the scope of the presently disclosed subject matter.

### EXAMPLES

### Example 1

Preparation of a Water Dispersible Flucytosine Capsule (500 mg), Sodium CMC with the Particle Size Between 177 to 420 Microns

A water-dispersible, granular formulation of Flucytosine was developed as described using dry granulation method. The following approaches were undertaken to prepare Flucytosine suspension:

(a) Flucytosine, microcrystalline cellulose, corn starch, sodium CMC, flavor, aspartame and magnesium stearate were added to the container and mixed well. Sodium CMC powder was passed through sieve 40 (420 microns) and then sieve 80 (177 microns) and the top fraction was collected from sieve 80 to manufacture the batch.

(b) Size “00” capsules were filled with the prepared powder composition.

Table 3 lists the composition of Flucytosine capsule. Each size 00 capsule contain following amounts:

TABLE 3
Composition of flucytosine granules for suspension

| | mg amount per | % amount per |
|---|---|---|
| Excipients | capsule | capsule |
| Flucytosine | 500 mg | 86.2 |
| Microcrystalline cellulose | 25 mg | 4.3 |
| Raspberry flavor | 2.5 mg | 0.43 |
| Aspartame | 2.5 mg | 0.43 |
| Corn starch | 15 mg | 2.6 |
| Sodium CMC (above 180 micron) | 25 mg | 4.3 |
| Magnesium stearate | 10 mg | 1.72 |

Drug composition equivalent to 500 mg Flucytosine was taken out and placed in 10 mL water. Upon simple stirring for 1 minute, it produced a nice suspension. Microcrystalline cellulose and sodium CMC provide enough suspendability to the suspension so that, particles did not settle immediately in the solution. Flavor and taste was also good.

### Example 2

Preparation of a Water Dispersible Flucytosine Capsule (500 mg), Sodium CMC Below 177 Microns Particle Size

A water-dispersible, granular formulation of Flucytosine was developed as described. The following method was undertaken in an attempt to prepare Flucytosine suspension:

(a) Flucytosine, microcrystalline cellulose, corn starch, sodium. CMC, Flavor, Aspartame and magnesium stearate were added to the container and mixed well. Sodium CMC were passed through sieve 80 (177 microns) and bottom fraction was collected and used for the batch.

(b) Size “00” capsules were filled using prepared powder composition.

Table 4 lists the composition of Flucytosine capsule. Each size 00 capsule contain following amounts:

TABLE 4
Composition of flucytosine granules for suspension.

| | mg amount per | % amount per |
|---|---|---|
| Excipients | capsule | capsule |
| Flucytosine | 500 mg | 86.2 |
| Microcrystalline cellulose | 25 mg | 4.3 |
| Raspberry flavor | 2.5 mg | 0.43 |
| Aspartame | 2.5 mg | 0.43 |
| Corn starch | 15 mg | 2.6 |
| Sodium CMC (below 177 micron) | 25 mg | 4.3 |
| Magnesium stearate | 10.0 mg | 1.72 |

Granules equivalent to 500 mg Flucytosine were taken out and placed in 10 mL water. Upon simple stirring for 1 minute, it produced suspension, but small lumps were observed. Microcrystalline cellulose and sodium CMC provided enough viscosity and suspendability to the suspension but did not give good homogeneous suspension compared to Example 1. Also, flow property of powder was not as good as in Example 1.

### Example 3

### Preparation of a Water Dispersible Flucytosine (500 mg)

A water-dispersible, granular formulation of Flucytosine was developed as described. The following method was undertaken to prepare Flucytosine suspension formulation:

(a) Flucytosine, microcrystalline cellulose, colloidal silicon dioxide, starch 1500 and hydroxypropyl methylcellulose were mixed well.

(b) about 10% water was added to above mixture for preparation of granules. The granules were dried at 40° C.

(c) Flavor, magnesium stearate and Aspartame were added to the granules and mixed well.

(d) Granules were filled in pouches.

Table 5 show the composition of Flucytosine granule formulation. The table also lists the % of each ingredient per pouch.

TABLE 5
Composition of Flucytosine formulation for suspension.

| Excipients | mg amount per pouch | % amount per pouch |
|---|---|---|
| Flucytosine | 500 mg | 77.16 |
| Microcrystalline cellulose | 50 mg | 7.72 |
| Artificial cherry flavor | 5 mg | 0.77 |
| Aspartame | 10 mg | 1.54 |
| Starch 1500 | 20 mg | 3.09 |
| Hydroxypropyl | 50 mg | 7.72 |
| methylcellulose | | |
| Colloidal silicon dioxide | 5 mg | 0.77 |
| Magnesium Stearate | 8 mg | 1.23 |

Granules equivalent to 500 mg Flucytosine were taken out from the pouch and were placed in 10 mL water. Upon simple stirring, it produced a good suspension but not good as Example 1. Microcrystalline cellulose, hydroxylpropyl methyl cellulose and colloidal silicon dioxide provided enough suspendability to the suspension so that particles did not settled immediately in the solution.

### Example 4

Preparation of a Water Dispersible Flucytosine with Amphotericen B Capsule

A water-dispersible, granular formulation of Flucytosine and amphotericin B was developed as described. The following wet granulation method was used to prepare Flucytosine and amphotericin B suspension:

(a) Flucytosine, Amphotericin B, microcrystalline cellulose, corn starch and sodium CMC were mixed well.

(b) about 10% water was added to above mixture for preparation of granules. The granules were dried at 40° C.

(c) Flavor and Aspartame were added to the granules and mixed well.

(d) Size 00 capsules were filled using prepared granules.

Table 6 show the composition of Flucytosine capsule. Each size 00 capsule contain following amounts:

TABLE 6
Composition of flucytosine granules for suspension.

| | mg amount | |
|---|---|---|
| Excipients | per capsule | % amount per capsule |
| flucytosine | 250 mg | 60.2 |
| Amphotericin B | 50 mg | 12.0 |
| Microcrystalline cellulose | 25 mg | 6.0 |
| Orange flavor | 5 mg | 1.3 |
| Aspartame | 5 mg | 1.3 |
| Corn starch | 20 mg | 4.8 |
| Sodium CMC | 50 mg | 12.0 |
| Magnesium stearate | 10 mg | 2.4 |

Contents of capsule were emptied out and placed in 10 mL water. Upon simple stirring, it produced a nice suspension. Microcrystalline cellulose and sodium CMC provided enough suspendability to the suspension so that particles did not settled immediately in the solution.

### Example 5

Preparation of a Water Dispersible Flucytosine with Amphotericen B and Ketoconazole Capsule

A water-dispersible, granular formulation of Flucytosine, amphotericin B and ketoconazole was developed as described. The following method was used to prepare Flucytosine suspension:

(a) Flucytosine, Amphotericin B, Ketoconazole, microcrystalline cellulose, corn starch and sodium CMC were mixed well.

(b) about 10% water was added to above mixture for preparation of granules. The granules were dried at 40″ C.

(c) Flavor and Aspartame were added to the granules and mixed well.

(d) Size 00 capsules were filled using prepared granules.

Table 7 shows the composition of Flucytosine capsule. Each size 00 capsule contain following amounts:

TABLE 7
Composition of flucytosine, amphotericin and ketoconazole granules
for suspension.

| Excipients | mg amount per capsule | % amount per capsule |
|---|---|---|
| Flucytosine | 250 mg | 44.05 |
| Amphotericin B | 25 mg | 4.41 |
| Ketoconazole | 125 mg | 22.03 |
| Microcrystalline | 50 mg | 8.81 |
| cellulose | | |
| Artificial cherry flavor | 2.5 mg | 0.44 |
| Aspartame | 5 mg | 0.88 |
| Corn starch | 50 mg | 8.81 |
| Sodium CMC | 50 mg | 8.81 |
| Magnesium stearate | 10 mg | 1.76 |

Contents of capsule were taken out and placed in 10 mL water. Upon simple stirring, it produced a nice suspension. Microcrystalline cellulose and sodium CMC provided enough suspendability to the suspension so that particles did not settle immediately in the solution.

### Dissolution of Granules

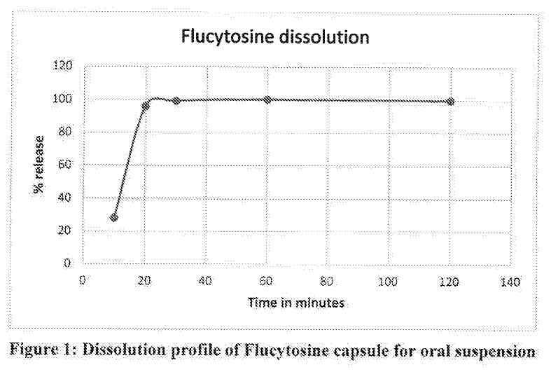

Dissolution studies in water were performed for Flucytosine size 0 capsules. USP II apparatus were used and UV-visible spectrometer was used for quantification of Flucytosine from dissolution. FIG. 1 shows the dissolution profile of flucytosine where 100 percent drug dissolved in 20 minutes. The solubility of flucytosine in water is reported to be 15 mg/mL. Thus, for a dose of 500 mg flucytosine, 33.3 mL water is needed to dissolve the drug. During administration of this drug to the patient, formulation containing 500 mg flucytosine is added to about 10 mL water. It means, out of 500 mg flucytosine, 150 mg drug dissolved in water and the remaining drug was suspended. When we combine other antifungal agents in this formulation, the dissolved fraction of flucytosine will change.

### SUMMARY

In summary, it is concluded that formulations of flucytosine, which features water-dispersible pharmaceutical compositions comprising the active ingredient flucytosine with optionally other antifungal agents in granular or powder form. The particle size of suspending agent chosen is 177 to 420 micron that allows a “quick” formation of a palatable liquid suspensions suitable for pediatric or geriatric dosing and person having swallowing difficulties. The selected particle size of the suspending agent(s) allows rapid wetting of the suspending agent and quick formation of a stable suspension. There are no sedimentation issues and provide a stable suspension. It also gives better flow to the powder and avoid the granulation step. The granules can be sprinkled on food items such as Jell-O, yogurt, apple sauce etc. When the granules are filled in a capsule, an adult patient may prefer to take the capsule as is with a glass of water. The same capsule can be opened, and the contents can be suspended in ˜5-10 mL water for pediatric/geriatric patients. The granules of drug compositions can be prepared using a dry granulation or a wet granulation method.

### REFERENCES

1. Manolakaki, D.; Velmahos, G.; Kourkoumpetis, T.; Chang, Y.; Alain, H. B.; De Moya, M. M.; Mylonakis, E. (2010), “Candida infection and colonization among trauma patients”. Virulence. 1 (5): 367-75.
2. Pfaller, M. A.; Diekema, D. J.; Colombo, A. L.; Kibbler, C.; Ng, K. P.; Gibbs, D. L.; Newell, V. A, (2006). “Candida rugosa, an emerging fungal pathogen with resistance to azoles: geographic and temporal trends from the ARTEMIS DISK antifungal surveillance program”. J. Clin. Microbiol. 44 (10): 3578-82.
3. Enfert C, Hube B (editors) (2007). Candida: Comparative and Functional Genomics. Caister Academic Press. ISBN 978-1-904455-13-4).
4. Qasem Jaber, Farmen Raymond H, and Phelps Kenneth, Topical formulations of Flucytosine, U.S. Pat. No. 9,314,524, Calla Therapeutics LLC.
5. Susan, Welsh and Richard D. wood, Novel topical formulations of Flucytosine and uses thereof, US 20090068287A1, Camargo Pharmaceutical services LLC.

That which is claimed:

1. A quick water-dispersing pharmaceutical composition comprising:
(i) about 50 weight % to about 95 weight % Flucytosine;
(ii) about 0.1 weight % to about 20 weight % of a suspending agent(s) with particle size 177 to 420 microns;
(iii) about 0.1 weight % to about 30 weight % of a diluent(s);
(iv) about 0.01 weight % to about 5 weight % of a sweetening agent(s);
(v) optionally about 0.01 weight % to about 5 weight % of a flavoring agent
(vi) optionally up to about 20 weight % of one or more of an additional excipients and adjuvants,
wherein the sum of (i)-(vi) is 100%; wherein the pharmaceutical composition is in the form of granules or a powder and the pharmaceutical composition is prepared by using the wet granulation method.

2. A quick water-dispersing pharmaceutical composition comprising:
(i) about 10 weight % to about 95 weight % combination of Flucytosine
(ii) about 10 weight % to about 95 weight % other anti fungal agent or a combination of other antifungal agents.
(iii) about 0.1 weight % to about 20 weight % of a suspending agent(s) with particle size 177 to 420 microns;
(iv) about 0.1 weight % to about 30 weight % of a diluent(s);
(v) about 0.01 weight % to about 5 weight % of one or more of a sweetening agent(s);
(vi) optionally about 0.01 weight % to about 5 wt. % of a flavoring agent
(vii) optionally up to about 20 weight % of one or more of an additional excipients and adjuvants,
wherein the sum of (i)-(vii) is 100%; wherein the pharmaceutical composition is in the form of granules or a powder and the pharmaceutical composition is prepared by using the wet granulation method.

3. A quick water-dispersing pharmaceutical composition comprising:
(i) about 10 wt. % to about 95 weight % combination of Flucytosine
(ii) optionally about 10 wt. % to about 95 weight % other antifungal agent or a combination of other antifungal agents.
(iii) about 0.1 wt. % to about 20 weight % of a suspending agent(s) with particle size 177 to 420 microns;
(iv) about 0.1 weight % to about 30 weight of a diluent(s);
(v) about 0.01 weight % to about 5 weight % of one or more of a sweetening agent(s);
(vi) optionally about 0.01 weight % to about 5 wt. % of a flavoring agent
(vii) optionally up to about 20 weight % of one or more of an additional excipients and adjuvants,
wherein the sum of (i)-(vii) is 100%; wherein the pharmaceutical composition is in the form of granules or a powder and the pharmaceutical composition is prepared by using the dry granulation method.

4. The pharmaceutical composition of claim 2, wherein other anti-fungal agents are amphotericin B, ketoconazole, itraconazole, fluconazole, voriconazole, Posaconazole, isavuconazole, clotrimazole, micafungin, anidulafungin, griseofulvin, terbinafine, efinaconazole, econazole, miconazole, terconazole, butoconazole, ciclopirox olamine, tolnaftate, nystatin and many more.

5. The pharmaceutical composition of claim 3, wherein other anti-fungal agents are amphotericin B, ketoconazole, itraconazole, fluconazole, voriconazole, Posaconazole, isavuconazole, clotrimazole, micafungin, anidulafungin, griseofulvin, terbinafine, efinaconazole, econazole, miconazole, terconazole, butoconazole, ciclopirox olamine, tolnaftate, nystatin and many more.

6. The quick water-dispersing pharmaceutical composition of claim 1 is administered by suspending in the aqueous phase for formulation or sprinkling on food or administering as an intact capsule.

7. The quick water-dispersing pharmaceutical composition of claim 2 is administered by suspending in the aqueous phase for formulation or sprinkling on food or administering as an intact capsule.

8. The quick water-dispersing pharmaceutical composition of claim 3 is administered by suspending in the aqueous phase for formulation or sprinkling on food or administering as an intact capsule.

9. The quick water-dispersing pharmaceutical composition of claim 1 is supplied as granules or powder in a hard capsule, in a packet or in a pouch.

10. The quick water-dispersing pharmaceutical composition of claim 2 is supplied as granules or powder in a hard capsule, in a packet or in a pouch.

11. The quick water-dispersing pharmaceutical composition of claim 3 is supplied as granules or powder in a hard capsule, in a packet or in a pouch.

12. The quick water-dispensing pharmaceutical composition of claim 1 disperse in water within 5 seconds to 5 minutes.

13. The quick water-dispensing pharmaceutical composition of claim 2 disperse in water within 5 seconds to 5 minutes.

14. The quick water-dispensing pharmaceutical composition of claim 3 disperse in water within 5 seconds to 5 minutes.

15. The quick water-dispersing pharmaceutical composition of claim 1, wherein suitable suspending agent(s) is selected from a group comprising microcrystalline cellulose, colloidal silicon dioxide, sodium carboxymethyl cellulose, hydroxypropyl methylcellulose, hydroxylpropyl cellulose, methyl cellulose, sodium alginate, acacia, xantham gum, carbomer, bentonite acrylic acid polymer, methacrylate polymers and mixtures thereof.

16. The quick water-dispersing pharmaceutical composition of claim 2, wherein suitable suspending agent(s) is selected from a group comprising microcrystalline cellulose, colloidal silicon dioxide, sodium carboxymethyl cellulose, hydroxypropyl methylcellulose, hydroxylpropyl cellulose, methyl cellulose, sodium alginate, acacia, xantham gum, carbomer, bentonite acrylic acid polymer, methacrylate polymers and mixtures thereof.

17. The quick water-dispersing pharmaceutical composition of claim 3, wherein suitable suspending agent(s) is selected from a group comprising microcrystalline cellulose, colloidal silicon dioxide, sodium carboxymethyl cellulose, hydroxypropyl methylcellulose, hydroxylpropyl cellulose, methyl cellulose, sodium alginate, acacia, xantham gum, carbomer, bentonite acrylic acid polymer, methacrylate polymers and mixtures thereof.

18. The quick water-dispersing pharmaceutical composition of claim 1, wherein the additional excipients and adjuvants are selected from binders, carriers, lubricants, flow control agents, and crystallization retarders.

19. The quick water-dispersing pharmaceutical composition of claim 2, wherein the additional excipients and adjuvants are selected from binders, carriers, lubricants, flow control agents, and crystallization retarders.

20. The quick water-dispersing pharmaceutical composition of claim 3, wherein the additional excipients and adjuvants are selected from binders, carriers, lubricants, flow control agents, and crystallization retarders.
